(19) United States
(12) Patent Application Publication
(10) Pub. No.: US 2022/0288228 A1
(43) Pub. Date: Sep. 15, 2022

(54) METHODS OF CANCER TREATMENT BY DELIVERY OF siRNAs AGAINST BCLXL AND MCL1 USING A POLYPEPTIDE NANOPARTICLE

(71) Applicant: Sirnaomics, Inc., Gaithersburg, MD (US)

(72) Inventors: David EVANS, Gaithersburg, MD (US); Vera SIMONENKO, Gaithersburg, MD (US)

(21) Appl. No.: 17/694,224

(22) Filed: Mar. 14, 2022

Related U.S. Application Data
(60) Provisional application No. 63/160,810, filed on Mar. 14, 2021.

(51) Int. Cl. A61K 47/69; A61K 33/243; A61K 31/713; C12N 15/113

(52) U.S. Cl. CPC A61K 47/6929; A61K 33/243; A61K 31/713; C12N 15/113

(57) ABSTRACT

Compositions and methods are provided for the silencing of the BCLxL and MCL1 genes. Specifically, siRNA compositions are provided that contain siRNA molecules that target the BCLxL and MCL1 genes. Methods for using these compositions for treating cancer also are provided.

### CROSS-REFERENCE TO RELATED PATENT APPLICATIONS

This application claims the benefit of and priority to U.S. Provisional Patent Application No. 63/160,810, filed Mar. 14, 2021, the contents of which are incorporated herein by reference in their entirety.

### BACKGROUND

Proteins in the BCL-2 family are regulators of the intrinsic apoptosis pathway. They contain one to four BCL-2 homology motifs (BH1-BH4) and can be divided into pro-apoptotic and antiapoptotic proteins. The anti-apoptotic multidomain members (BH1-BH4) include BCL-2, BCLxL, BCL-w, BFL-1/A1 and MCL-1, and these proteins function to counteract the pore-forming activity of the pro-apoptotic multidomain proteins BAX and BAK which permeabilize the mitochondrial outer membrane. Following various stress signals, the BH3-only proteins either neutralize the anti-apoptotic proteins or directly activate effector proteins BAX and BAK which will eventually lead to apoptosis in cells. Campbell and Tait, Open Biol. 8: 180002 (2018); Quayle et al., Oncotarget 8:88670-88688 (2017). Cancer cells can evade apoptosis, triggered by oncogenesis or drug treatment, by overexpressing the BCL-2 antiapoptotic proteins. Hanahan and Weinberg, Cell 144:646-674 (2011).

Specific small molecule inhibitors have been developed against BCLxL (ABT-199/venetoclax; Souers et al., Nat. Med. 19:202-208 (2013)) and MCL1 (S63845; Li et al., Leukemia 33L:262-266 (2019)). Combining these small molecule inhibitors has shown therapeutic benefit in treating a number of cancer types including cervical cancer (Rahman et al, Biochem. Biophys. Reports, 22:100756 (2020)), lung squamous cell carcinomas (Clare et al, Oncogene 37:4475-4488 (2018)) and head and neck cancer (Thomas et al., Oncotarget, 10:494-510 (2019)).

While small molecule inhibitors have been demonstrated to function to block both BCLxL and MCL1 and induce a therapeutic benefit there are issues with their use. For example ABT263 selectively inhibits BCL-2, BCLxL and BCL-w (Tse et al., Canc. Res. 68:3421-3428 (2008)) but induces thrombocytopenia as a consequence of its inhibition of BCLxL (Mason, et al., Cell 128:1173-1186 (2007); Zhang et al., Cell Death Differ. 14:943-951 (2007)).

It has also been shown that combining BCLxL and MCL1 siRNAs can inhibit ovarian tumors. (Brotin et al., Int. J. Cancer 126:885-895 (2010); WO2008/001156). A combination of BCLxL and MCL1 siRNAs has been shown to inhibit pancreatic tumors (Takahashi et al., Biochimica et Biophysica Acta 1833:2980-2987 (2013)). The apoptosis-inducing effect of simultaneous knock-down of BCLxL and MCL-1 is associated with translocation of Bax from the cytosol to the mitochondrial membrane, cytochrome c release, and caspase activation. These results demonstrated that BCLxL and MCL-1 play an important role in pancreatic cancer cell survival.

### SUMMARY OF THE INVENTION

Nanoparticle compositions are provided that contain a BCLxL-silencing amount of an siRNA molecule that targets BCLxL and an MCL1-silencing amount of an siRNA molecule that targets MCL1. The siRNA that targets BCLxL may be selected from the group consisting of molecules having a sequence denoted by SEQ ID NOs:1-8 and the siRNA that targets MCL1 may be selected from the group consisting of molecules having a sequence denoted by SEQ ID NOs:9-13. In one embodiment the siRNA that targets BCLxL is selected from the group consisting of SEQ ID NOs:1, 4, 5, 7 and 8 and the siRNA that targets MCL1 is selected from the group consisting of SEQ ID NOs:10-13. In a specific embodiment, the siRNA that targets BCLxL is SEQ ID NO:5, which optionally may be combined with SEQ ID NO:10 or SEQ ID NO:12 as an siRNA that targets MCL1.

The nanoparticle may comprise an HKP, and the HKP may be, for example, HKP(+H).

In specific embodiments, the ratio of the siRNA that targets MCL1 to the siRNA that targets BCLxL is about 1:1 or more. On other embodiments the ratio may be from about 1:1 to about 3:1, from about 2:1 to about 3:1, or about 2:1 or about 3:1.

Also provided are methods of treating a cancer in a subject suffering from the cancer, in which an effective amount of a nanoparticle composition as described above is administered to the subject of a composition. The cancer may be, for example head and neck cancer, bladder cancer, pancreatic cancer, cholangiocarcinoma, lung cancer (NSCLC and SCLC), colon cancer, glioblastoma, breast cancer, gastric adenocarcinomas, prostate cancer, ovarian carcinoma, cervical cancer, AML, ALL, myeloma or non-Hodgkins lymphoma. In these methods the composition may be delivered systemically or intratumorally.

Further provided are methods of treating cancer in a subject, in which the nanoparticle composition as described above is administered together with an effective amount of a chemotherapy drug. Examples of suitable chemotherapy drugs are platinum-containing drugs such as cisplatin, oxaloplatin, or carboplatin.

### BRIEF DESCRIPTION OF THE DRAWINGS

FIG. 1 shows a graph demonstrating the efficacy of silencing the BCLxL gene by siRNA molecules BCLxL#1, #4, #5, #6, #7 and #8 in FaDu cells.

FIG. 2 shows the activity of siRNA molecules hmMCL1_1, hmMCL1_2, hmMCL1_3 and hmMCL1_4 in silencing the MCL1 gene in FaDu cells.

FIG. 3 shows the ability of chimeric sequences to silence BCLxL and 2 respective genes.

FIG. 4 shows silencing of BCLxL in FaDu cells by 4 chimeras.

FIG. 5 (a)-(e) show results of a nanoparticle assessment at a variety of flow rates. Total Flow Rate (TFR) was varied and the effect of flow rate on particle size evaluated by measuring resulting particle size. PDI=polydispersity index.

FIG. 6 shows that mixing at 10 mgs/ml produced a highly uniform nanoparticle.

FIG. 7 shows that administration of siRNAs in the same nanoparticle silences both BCLxL and MCL1 within the same cell that takes up the siRNA nanoparticle.

FIG. 8 shows the effect of administering BCLxL and MCL1 siRNAs alone or in combination at varying concentrations in HTB9 (bladder cancer) cells.

FIG. 9 shows the effect of administering BCLxL and MCL1 siRNAs alone or in combination at varying concentrations in UMUC-3 cells (another bladder cancer cell line). The same process was used but exposure was only 72h to siRNAs prior to measuring cell viability.

FIG. 10 shows the effect of administering BCLxL and MCL1 siRNAs alone or in combination against pancreatic tumor cells.

FIG. 11 shows the effect of administering BCLxL and MCL1 siRNAs alone or in combination at varying concentrations in H&N Cancer cells.

FIG. 12 shows the effect of combining siRNAs against MCL1 (Seq#2) with siRNA against BCLxL (seq #5) at varying ratios.

FIG. 13 shows the effect of combining MCL1#4 with BCLxL#5.

FIG. 14 shows the results of using ratios of 2:1 and 3:1 for several mixtures of MCL1 siRNA with BCLxL siRNAs.

FIG. 15 shows the effects of combining the siRNAs with cisplatin in FaDu cells.

FIG. 16 shows that a combination of siRNAs against BCLxL and MCL1 is able to inhibit xenografts of H&N cancer when administered intratumorally.

### DETAILED DESCRIPTION

Compositions and methods are provided for the silencing of the BCLxL and MCL1 genes. Specifically, siRNA compositions are provided that contain effective amounts of siRNA molecules that target the BCLxL and MCL1 genes by reducing expression of the protein products of those genes. Methods for using these compositions for treating cancer also are provided. Silencing BCLxL and MCL1 concomitantly using siRNA molecules inhibits the growth of several tumor types including bladder cancer and Head and Neck Cancer. Several siRNA sequences able to specifically and potently silence the BCLxL and MCL1 genes are provided. The sequences described below are the sense strands of blunt-ended double stranded RNA molecules. The skilled artisan will appreciate that the siRNA molecules contain the sense strand as shown as part of a duplex with its complementary sequence. Reference herein to the siRNA molecule of SEQ ID NO:X will be understood to refer to the duplex formed by the sense strand (SEQ ID NO:X) and the corresponding antisense strand.

As used herein, silencing a gene means reducing the concentration of the mRNA transcript of that gene such that the concentration of the protein product of that gene in a cell or tissue is reduced by at least 10%, at least 20%, at least 30%, at least 40%, at least 50%, at least 70%, at least 80% or at least 90% or more. Measurement of the reduction in protein concentration may be achieved using methods that are well known in the art, such as ELISA. The reduction in the concentration of the mRNA transcript may be achieved using methods well-known in the art such as quantitative RT-PCR.

Selection of BCLxL siRNA sequences

The sequences shown below are the sense strands of the blunt-ended 25-mer siRNA molecules used to silence the BCLxL gene.

RNA sequence sense strand

| | |
|---|---|
| BCLxL_1:, | CCUACAAGCUUUCCCAGAAAGGAUA (SEQ ID NO: 1) |
| BCLxL_2:, | CCCAGUGCCAUCAAUGGCAACCCAU (SEQ ID NO: 2) |
| BCLxL_3: | GGAGCCACUGGCCACAGCAGCAGUU (SEQ ID NO: 3) |
| BCLxL_4: | CGGGGCACUGUGCGUGGAAAGCGUA (SEQ ID NO: 4) |
| BCLxL_5: | GCGUGGAAAGCGUAGACAAGGAGAU (SEQ ID NO: 5) |
| BCLxL_6: | GCGUAGACAAGGAGAUGCAGGUAUU (SEQ ID NO: 6) |
| BCLxL_7: | CCUUGUGAAGAUGAUAUACUAUUUU (SEQ ID NO: 7) |
| BCLxL_8: | GGUGAAAGUGCAGUUCAGUAAUAAA (SEQ ID NO: 8) |

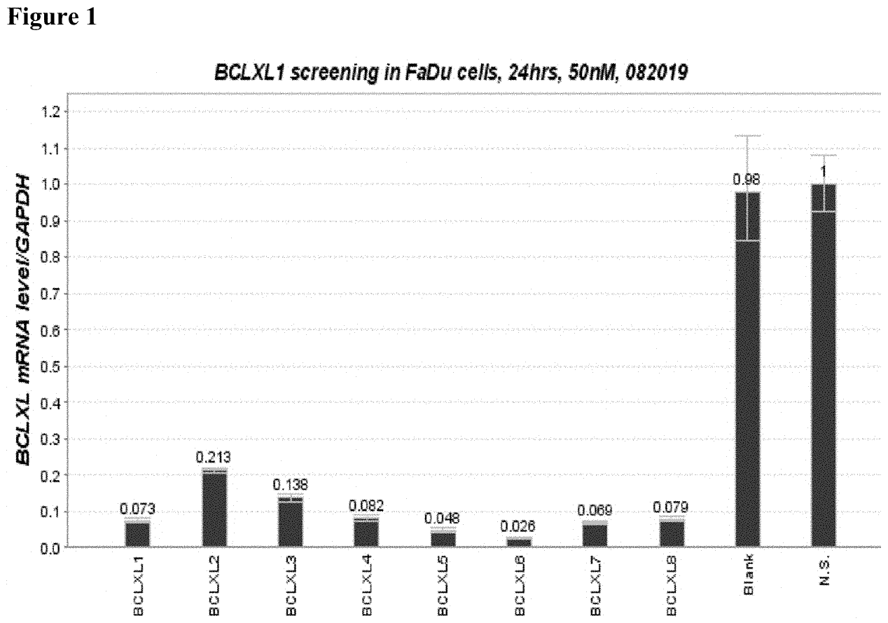

Sequences BCLxL#1, #4, #5, #6, #7 and #8 (SEQ ID NOs: 1, 4, 5, 6, and 7) all exhibited effective silencing in FaDu cells as shown in FIG. 1. FaDu cells are a cell line derived from a squamous cell carcinoma of the hypopharynx,)

Selection of MCL1 siRNA sequences

The 25-mer and 19-mer sequences shown below are the sense strands of the blunt-ended siRNA molecules used to silence the human MCL1 gene. These sequences are also common to murine MCL1V1.

RNA sequence sense strand

| | |
|---|---|
| hmMCL1_1 | 5′-GCUGGGAUGGGUUUGUGGAGUUCUU-3′ |
| | (SEQ ID NO: 9) |
| hmMCL1_2 | 5′-GCUAACAAGAAUAAAUACAUGGGAA-3′ |
| | (SEQ ID NO: 10) |
| hmMCL1_3 | 5′-GCAACCACGAGACGGCCUU-dTdT-3′ |
| | (SEQ ID NO: 11) |
| hmMCL1_4 | 5′-GGGAUGGGUUUGUGGAGUU-dTdT-3′ |
| | (SEQ ID NO: 12) |
| hmMCL1_5 | 5′-UAACACCAGUACGGACGGG-dTdT-3′ |
| | (SEQ ID NO: 13) |

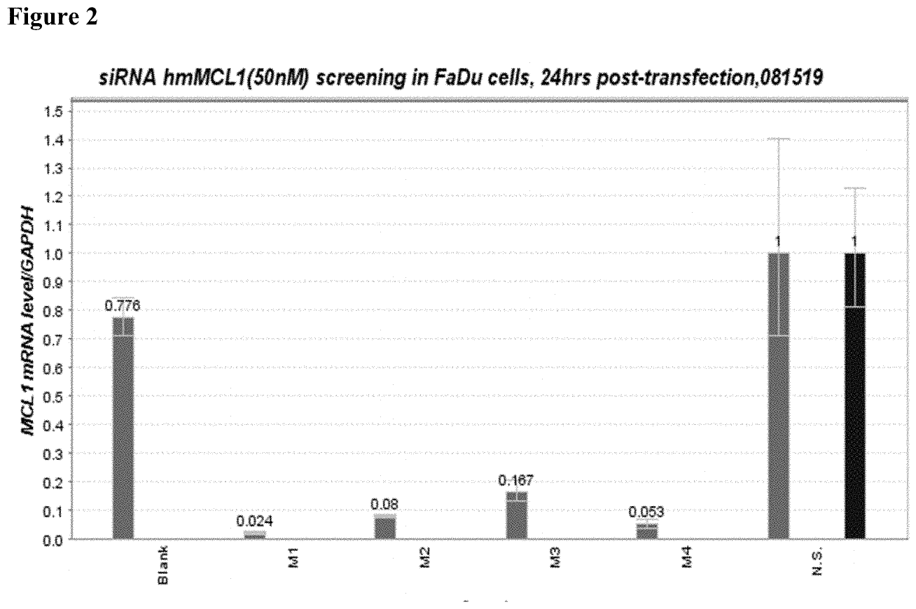

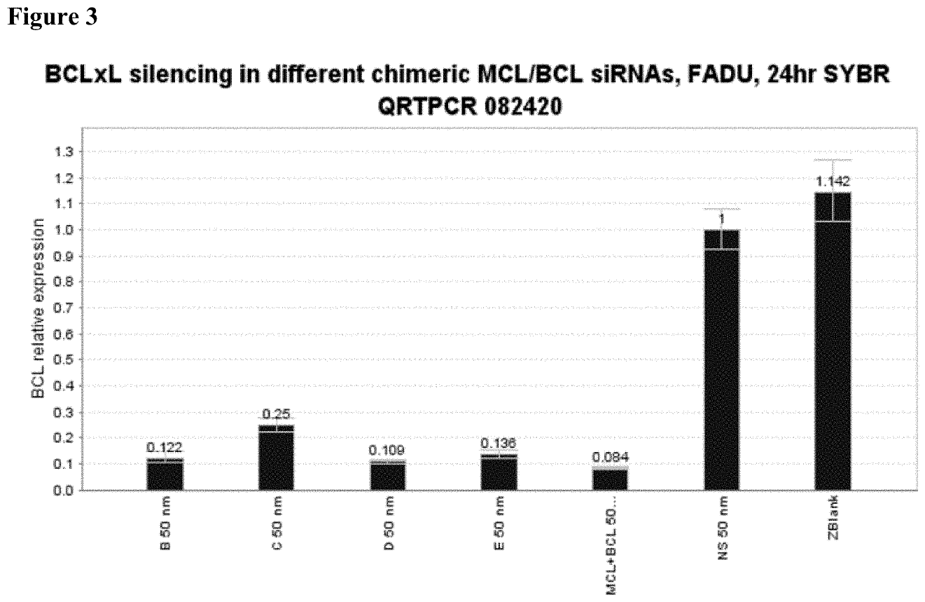

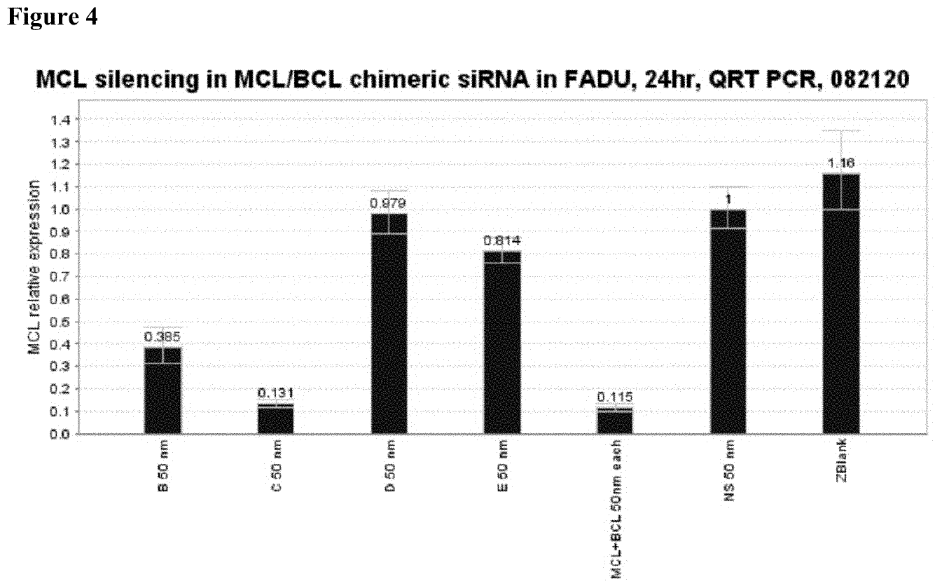

Sequence hmMCL1_5 (SEQ ID NO:13) has previously been described (Zhang et al., J. Biol. Chem., 277:37430-37438 (2002)). As shown in FIG. 2, sequences hmMCL1_1, hmMCL1_2, hmMCL1_3 and hmMCL1_4 (SEQ ID NOs:1-4) showed excellent activity in silencing the MCL1 gene in FaDu cells.

In each of these siRNA molecules, one or more of the nucleotides in either the sense or the antisense strand can be a modified nucleotide. Modified nucleotides can improve stability and decrease immune stimulation by the siRNAs. The modified nucleotide may be, for example, a 2′-O-methyl, 2′-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino or 2′-O-(N-methylcarbamate) ribonucleotide.

In addition, one or more of the phosphodiester linkages between the ribonucleotides may be modified to improve resistance to nuclease digestion. Suitable modifications include the use of phosphorothioate and/or phosphorodithioate modified linkages.

Formation of nanoparticles containing siRNAs targeting BCLxL and MCL1.

The siRNA molecules containing the described above advantageously are formulated into nanoparticles for administration to a subject. Various methods of nanoparticle formation are well known in the art. See, for example, Babu et al., IEEE Trans Nanobioscience, 15: 849-863(2016).

Advantageously, the nanoparticles are formed using one or more histidine/lysine (HKP) copolymers. Suitable HKP copolymers are described in WO/2001/047496, WO/2003/090719, and WO/2006/060182, the contents of each of which are incorporated herein in their entireties. HKP copolymers form a nanoparticle containing an siRNA molecule, typically 100-400 nm in diameter. HKP and HKP(+H) both have a lysine backbone (three lysine residues) where the lysine side chain ϵ-amino groups and the N-terminus are coupled to [KH₃]₄K (for HKP) or KH₃KH₄[KH₃]₂K (for HKP(+H). The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting BCLxL and an siRNA targeting MCL1 are mixed with one or more HKP polymers at a fixed flow rate. The flow rate can be varied to vary the size of the nanoparticles produced.

Thus, for example, an siRNA targeting BCLxL and an siRNA targeting MCL1 were mixed at 0.5 mg/ml with HKP(+H) using a PNI microfluidic mixer system (Precision Nanosystems, Inc., Vancouver, CA). Total Flow Rate (TFR) was varied and the effect of this flow rate on particle size was evaluated by measuring resulting particle size using a Malvern Nanosizer system (Malvern Panalytical Inc., Westborough, Mass.). The polydispersity index (PDI) is an indication of the amount of variation of the nanoparticles around the average size.

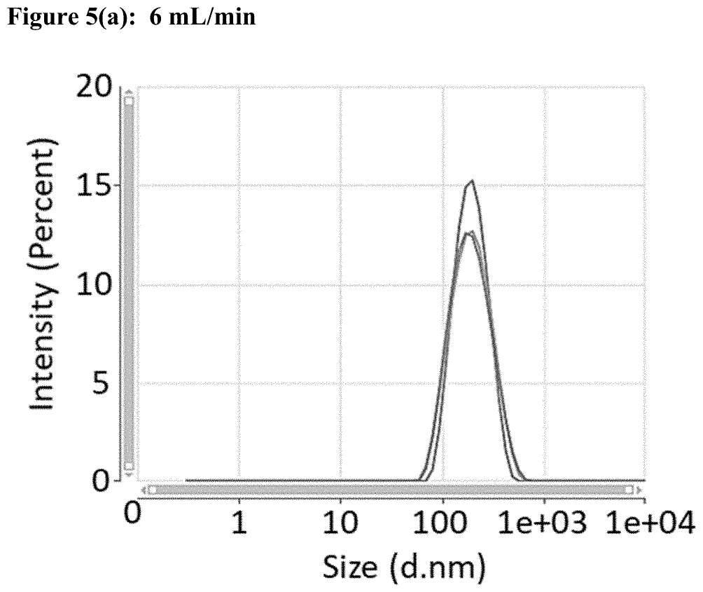

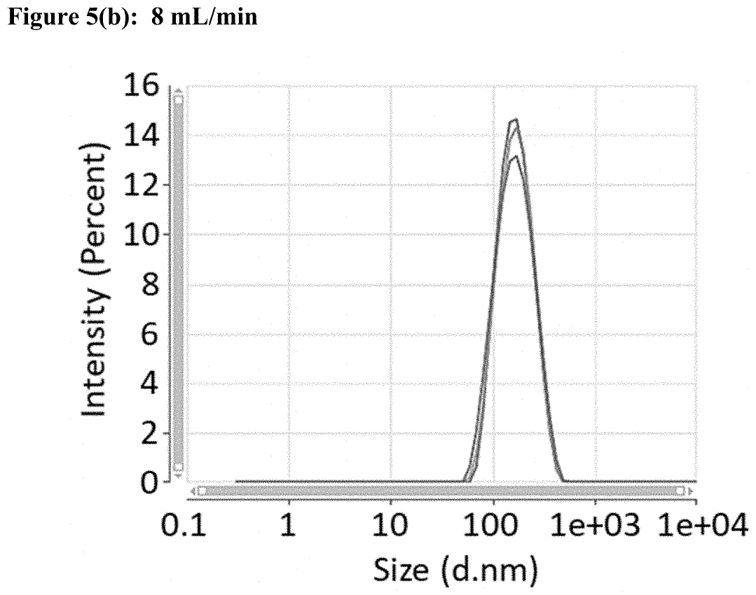

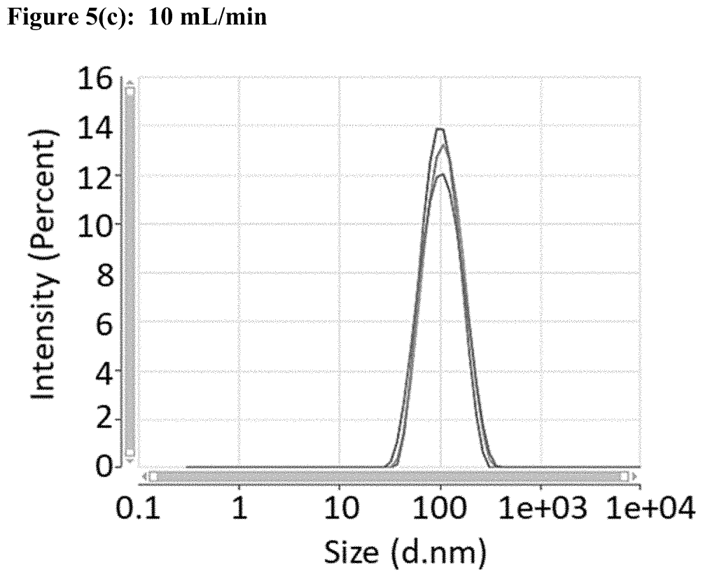

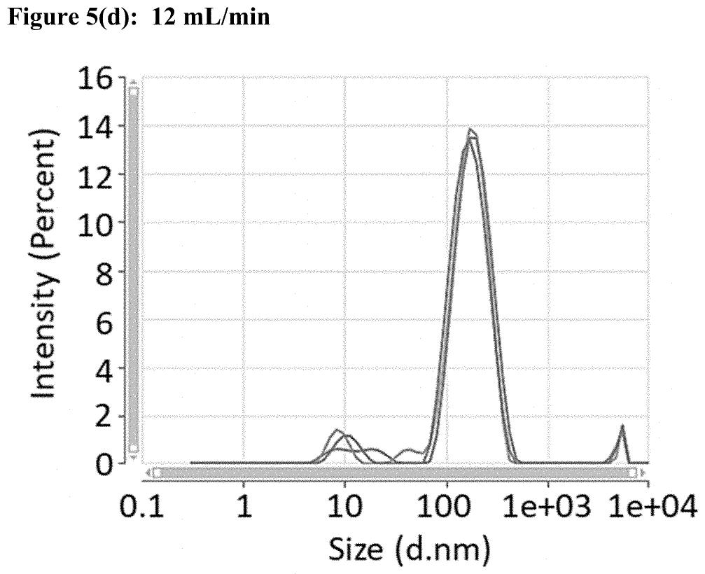

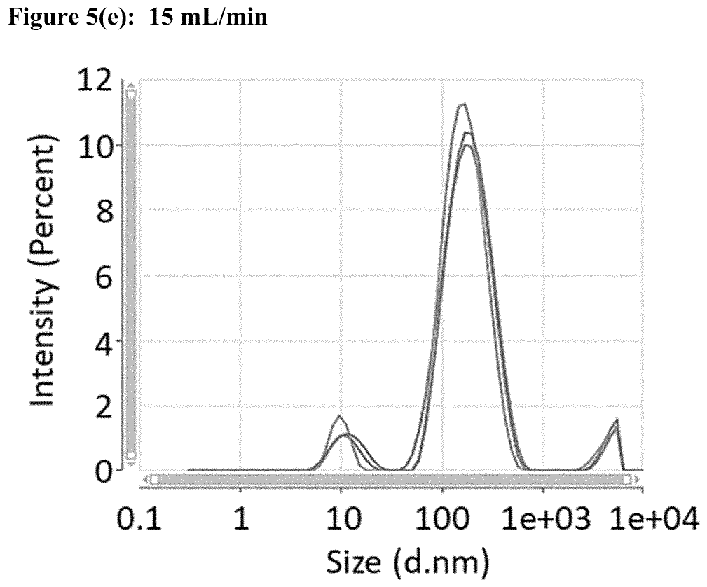

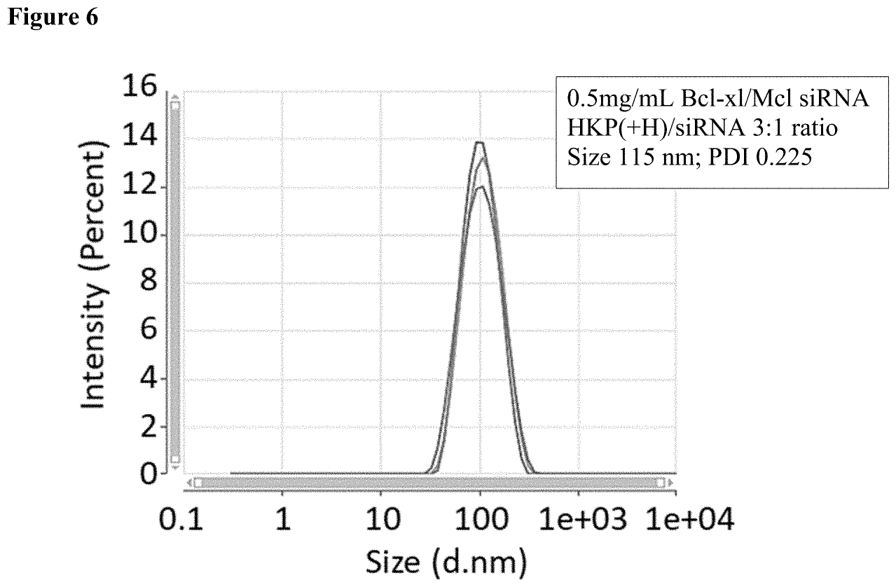

The resulting size dispersions of the nanoparticles are shown in FIG. 5 (a)-(e). Good uniformity was observed at flow rates of 10 ml/min and below. Mixing at 10 ml/ml produced a highly uniform nanoparticle between 102 nm (see Table 1) and 115 nm, as shown in FIG. 6.

TABLE 1

| | TFR | Size (nm) | PDI |
|---|---|---|---|
| 0.5 mg/mL siRNA | 6 mL/min | 176 | 0.164 |
| 3:1 ratio in water | 8 mL/min | 153 | 0.147 |
| HKP(+H) PPL1812 | 10 mL/min | 102 | 0.219 |
| hmBcl-xL | 12 mL/min | 147 | 0.442 |
| hmMcl1 | | Peak one: 9.57 | |
| | | Peak two: 186 | |
| | | Peak three: 5470 | |
| | 15 mL/min | 129 | 0.489 |
| | | Peak one 11.2 | |
| | | Peak two: 192 | |
| | | Peak three:5470 | |

Two siRNAs targeting BCLxL and MCL1 respectively were mixed at a 1:1 ratio and further mixed with HKP(+H) at a ratio of 3:1 (HKP(+H):siRNA) using a Precision Nanosystems Nanoassemblr microfluidic mixing device where the siRNAs were passed in one side of the mixer and HKP peptide was passed in the other side at a flow rate of 10 ml/min. The resulting nanoparticles showed a size of 102-115 nm with a PolyDispersity Index (PDI) of 0.219-0.225. The 2 siRNAs are incorporated in the nanoparticles equally, and when the siRNAs are administered in the nanoparticle they will each silence their respective gene sequence—silencing both BCLxL and MCL1 within the same cell that takes up the siRNA nanoparticle.

Determination of efficacy of the siRNA molecules

Depending on the particular target BCLxL and MCL1 RNA sequences and the dose of the nanoparticle composition delivered, partial or complete loss of function for the BCLxL and MCL1 RNAs may be observed. A reduction or loss of RNA levels or expression (either BCLxL and MCL1 RNA expression or encoded polypeptide expression) in at least 50%, 60%, 70%, 80%, 90%, 95% or 99% or more of targeted cells is exemplary. Inhibition of BCLxL and MCL1 RNA levels or expression refers to the absence (or observable decrease) in the level of BCLxL and MCL1 RNA or BCLxL and MCL1 RNA-encoded protein. Specificity refers to the ability to inhibit the BCLxL and MCL1 RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

Inhibition of target BCLxL and MCL1 RNA sequence(s) by the dsRNA agents of the invention also can be measured based upon the effect of administration of such dsRNA agents upon development/progression of a BCLxL and MCL1-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

Pharmaceutical compositions and methods of administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova

Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Determination of dosage and toxicity

Toxicity and therapeutic efficacy of the compositions may be determined by standard pharmaceutical procedures in cell cultures or experimental animals, e.g., by determining the LD50 (the dose lethal to 50 % of the population) and the ED₅₀ (the dose therapeutically effective in 50% of the population). The dose ratio between toxic and therapeutic effects is the therapeutic index and it can be expressed as the ratio LD₅/ED₅₀. Compounds advantageously exhibit high therapeutic indices

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

Suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

Methods of treatment

The compositions described herein may be used to treat proliferative diseases, such as cancer, characterized by expression, and particularly altered expression, of BCLxL and MCL1.

Exemplary cancers include liver cancer (e.g. hepatocellular carcinoma or HCC), lung cancer (e.g., NSCLC), colorectal cancer, prostate cancer, pancreatic cancer, ovarian cancer, cervical cancer, brain cancer (e.g., glioblastoma), renal cancer (e.g., papillary renal carcinoma), stomach cancer, esophageal cancer, medulloblastoma, thyroid carcinoma, rhabdomyosarcoma, osteosarcoma, squamous cell carcinoma (e.g., oral squamous cell carcinoma), melanoma, breast cancer, and hematopoietic disorders (e.g., leukemias and lymphomas, and other immune cell- related disorders). Other cancers include bladder, cervical (uterine), endometrial (uterine), head and neck, and oropharyngeal cancers. Advantageously, the cancer is head and neck cancer, bladder cancer, pancreatic cancer, cholangiocarcinoma, lung cancer (NSCLC and SCLC), colon cancer, glioblastoma, breast cancer, gastric adenocarcinomas, prostate cancer, ovarian carcinoma, cervical cancer, AML, ALL, myeloma or non-Hodgkins lymphoma.

The compositions may be administered as described above and, advantageously may be delivered systemically or intratumorally. The compositions may be administered as a monotherapy, i.e. in the absence of another treatment, or may be administered as part of a combination regimen that includes one or more additional medications. Advantageously, when used as part of a combination regimen that includes an effective amount of at least one additional chemotherapy drug. The chemotherapy drug may be, for example, a platinum-containing drug, such as cisplatin, oxaloplatin, or carboplatin.

### EXAMPLES

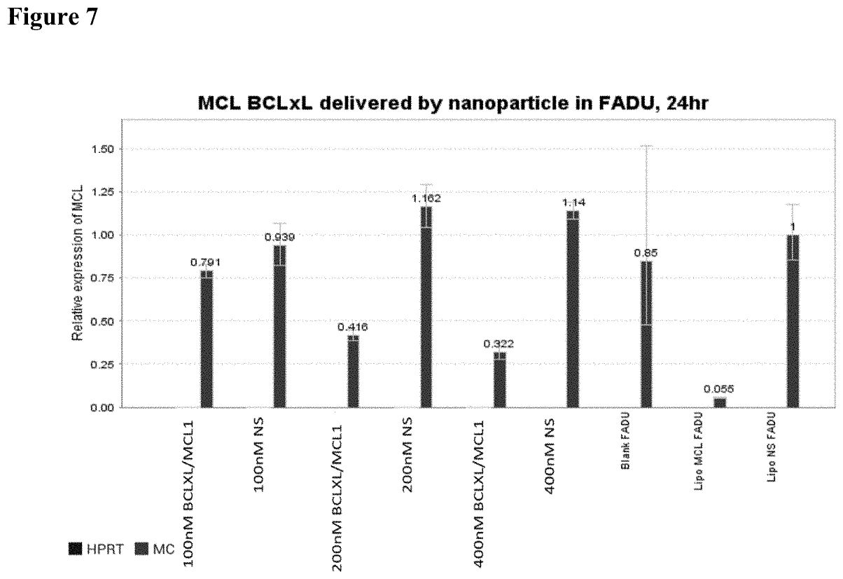

As shown in FIG. 7, a dose-dependent reduction of MCL1 was observed when the combination of BCLxL and MCL1 siRNAs was administered in the nanoparticles manufactured at 10 ml/min. Briefly FaDu cells were transfected by HKP(+H) nanoparticles formed using either BCLxL/MCL1 siRNAs or complexed with Non-Silencing (NS) siRNA as control. Other controls were (i) untreated cells, (ii) lipofectamine-delivered NS siRNA, and (iii) lipofectamine-delivered MCL1. After a 24h exposure to the nanoparticles the cells were recovered and used to measure MCL1 levels using quantitative RT-PCR. Methods of measuring mRNA levels in a cell using, for example, quantitative RT-PCR are well known in the art. Data were normalized to the Lipofectamine-delivered NonSilencing siRNA (Lipo NS; set as 1.0). Maximal silencing was demonstrated using Lipofectamine delivered siRNA (Lipo MCL). Untreated cells (Blank) were used to ensure that no significant effect of the controls was due to toxicity to the cells.

The presence of an HKP in a nanoparticle as described above provides properties that help uptake of the particle. The positively charged lysine residues in the HKP bind to the negatively charged backbone phosphate groups on siRNAs. This charge-charge attraction leads to spontaneous formation of nanoparticles when the siRNA(s) and the HKP are mixed together. The nanoparticles formed are typically below 200 nm in diameter, and the particle size may be varied in a microfluidic mixing system by varying the flow rate used during mixing, where faster flow rates in the mixing system result in smaller diameter nanoparticles (as low as 50 nm is feasible).

Upon administration to a subject suffering from cancer, the nanoparticles locate to tumors as a result of the Enhanced Permeability and Retention (EPR) effect. See Greish, Methods Mol

Biol. 624:25-37(2010). In particular, the nanoparticles may bind to specific receptors upregulated on many tumors (Neuropilin 1; NRP1); the particles are taken up into the tumor cells by micropinocytosis or receptor mediated entry where the nanoparticles enter the endosomes. Acidification of the endosomes occurs, protonating the basic histidines and creating a proton sponge effect, lysing the endosomal wall and releasing the siRNAs into the cytoplasm of the cell where they can inhibit the expression of the targeted genes.

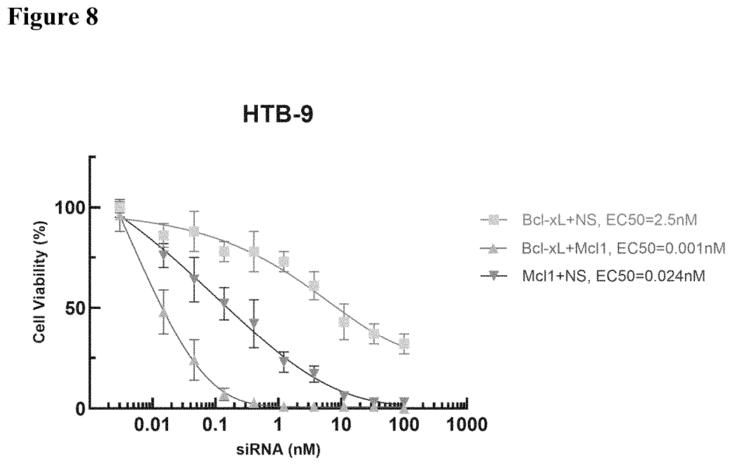

The combination of siRNAs delivered to bladder cancer cells (HTB-9 or UM-UC-3) in vitro shows surprisingly high additivity compared with each siRNA alone. The BCLxL and MCL1 siRNAs were delivered to the cells alone (combined with a control siRNA) or in combination using Lipofectamine RNAiMax at varying concentrations. See FIG. 8, which shows how the combination of siRNAs was significantly more potent than either individual siRNA.

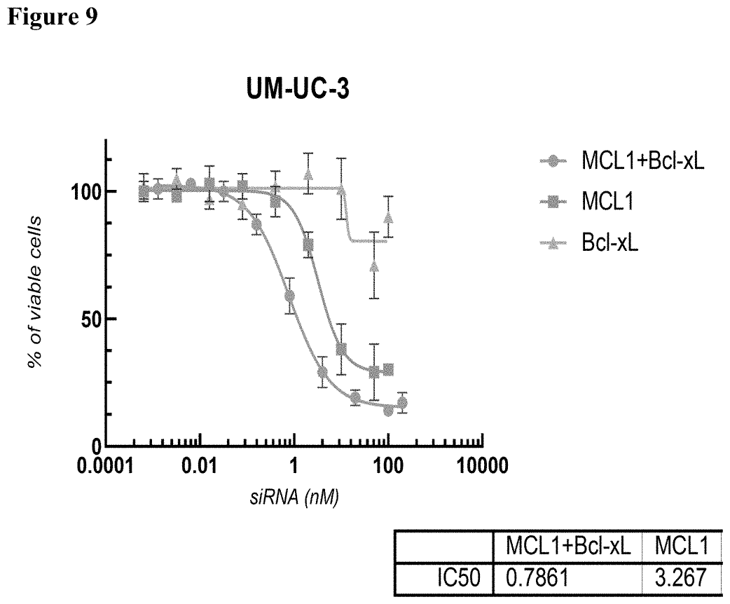

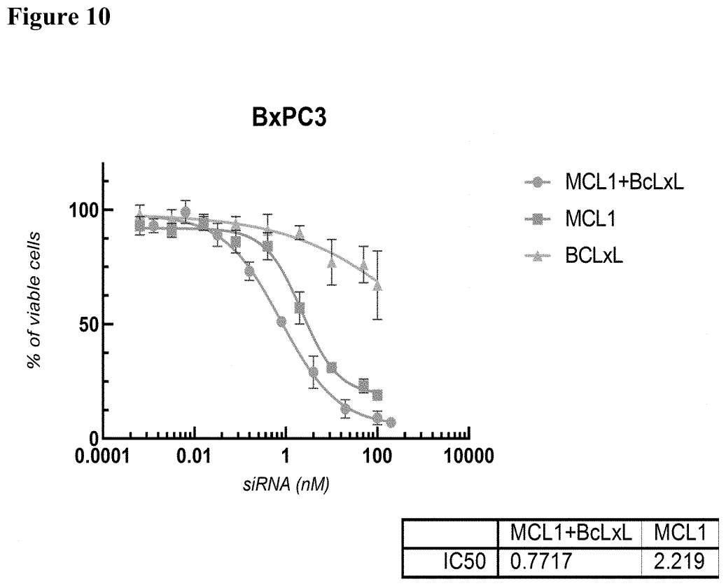

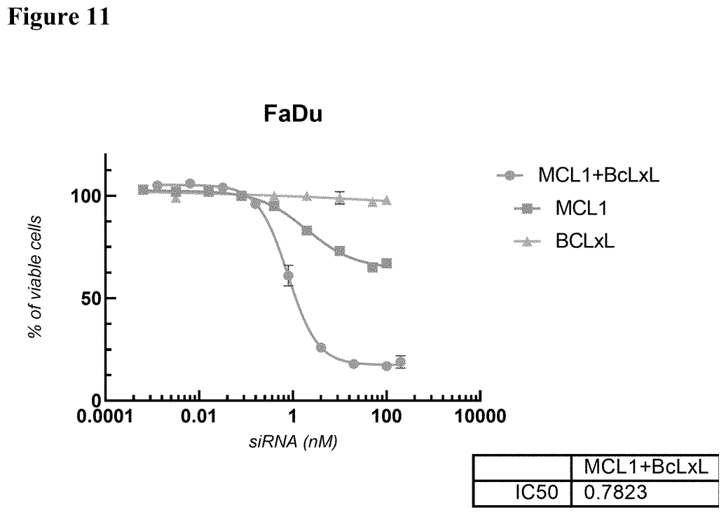

In HTB9 cells (FIG. 8) cell viability was monitored after 96h exposure to the siRNAs by using Cell Titer Glo2.0 (Promega). In UMUC-3 cells (another bladder cancer cell line) the same process was used but exposure was only 72h to siRNAs prior to measuring cell viability. See FIG. 9. The same 72h incubation time was used for pancreatic tumor cells (BxPC3) (FIG. 10) and Head and Neck (H&N) cancer cells (FaDu)(FIG. 11). The combination of BCLxL and MCL1 siRNAs showed activity against both the pancreatic tumor cells and the H&N cancer cells.

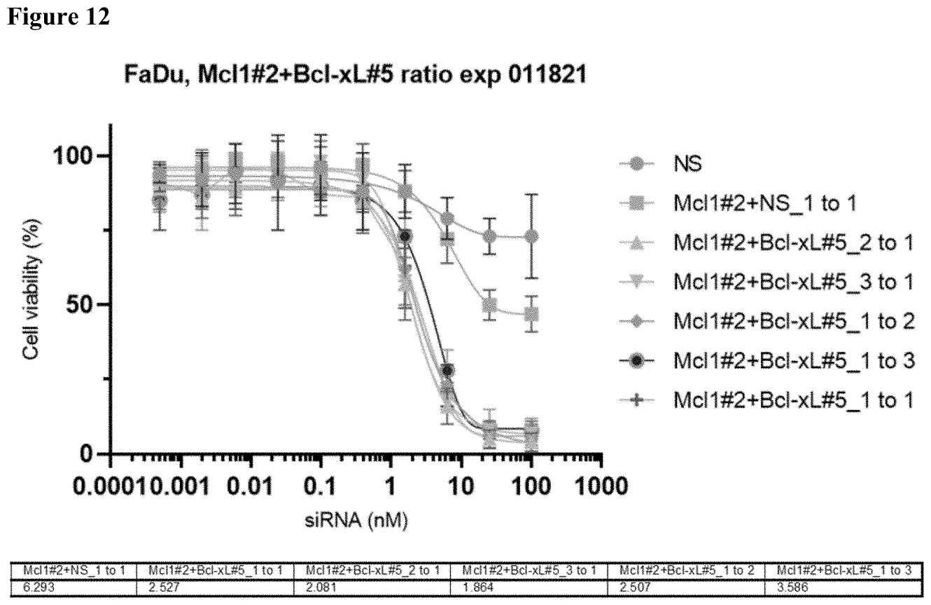

Multiple combinations of the siRNA sequences of SEQ ID NOs: 1-13 demonstrated activity. FIG. 12 shows data from an experiment where siRNAs against MCL1 (SEQ ID NO:10) and BCLxL (SEQ ID NO:5) were combined at varying ratios. NS=Non-silencing siRNA. As shown in FIG. 12, all ratios of this mixture showed relatively similar potency with identical maximal efficacy, killing ˜95% of the cells after a 72h exposure. The IC₅₀ values show that the optimal ratio was MCL1 SEQ ID NO:10 with BCLxL SEQ ID NO:5 at a ratio of 3:1. This mixture produced an IC₅₀ of 1.86 nM. For comparison purposes, a 1:1 ratio produced an IC₅₀ of 6.3 nM.

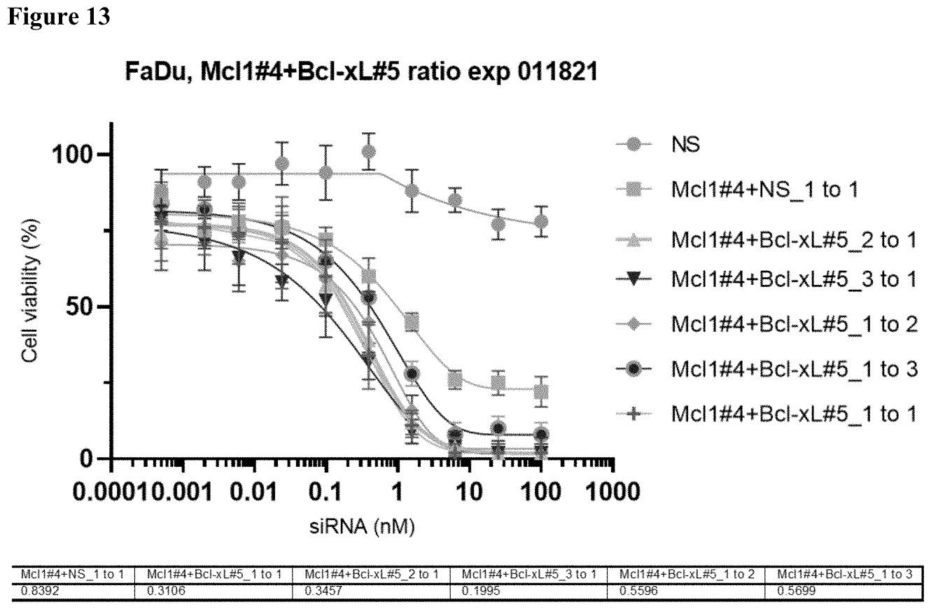

FIGS. 13 shows the results obtained when MCL1#4 (SEQ ID NO:12) was combined with BCLxL#5 (SEQ ID NO:5) under similar conditions. A much lower IC₅₀ was observed using a 1:1 ratio of these two sequences compared to the results shown in FIG. 12 for the combination of SEQ ID NOs: 4 and 10. Moreover, this result was improved even further by using a 3:1 ratio of MCL1 #2 (SEQ ID NO:10) and BCLxL #5 (SEQ ID NO:5) siRNAs which produced an IC₅₀ of 0.2 nM.

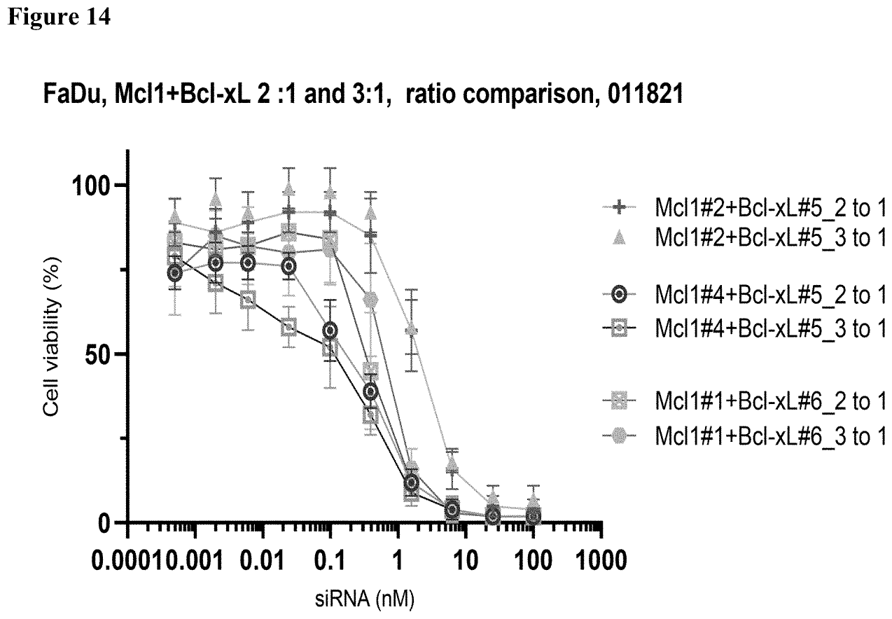

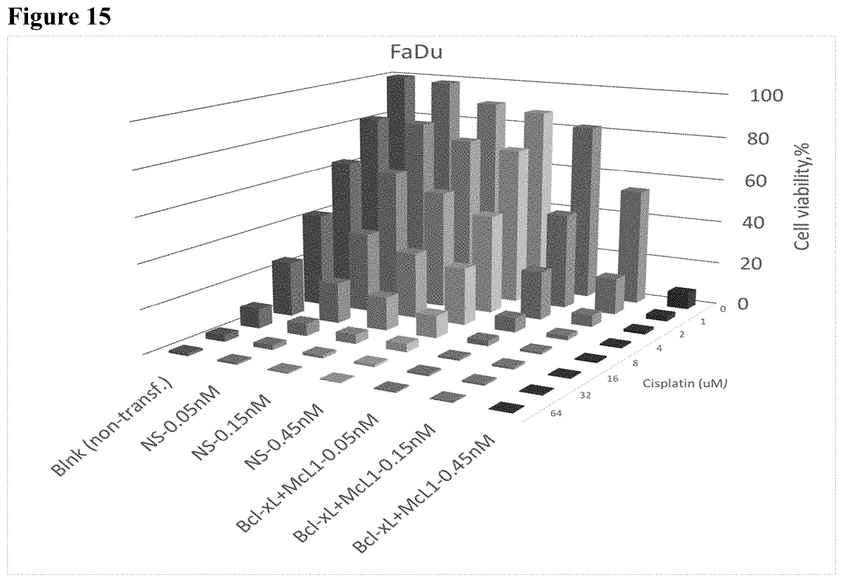

Further experiments evaluated the ratios of 2:1 and 3:1 of several mixtures of MCL1 siRNA with BCLxL siRNAs. As shown in FIG. 14 the mixture of MCL1#4 (SEQ ID NO:12) and BCLxL#5 (SEQ ID NO:5) gave the best potency and efficacy. In addition, as shown in FIG. 15, this combination of siRNAs was shown to potentiate the effect of cisplatin in treating H&N cancer cells (FaDu cells).

BCLxL and MCL1 siRNAs were mixed together in Lipofectamine RNAiMax and used to transfect FaDu H&N cancer cells. Final combined siRNA concentrations of 0.05 nM, 0.15 nM and 0.45 nM were compared with the effect of a non-silencing (NS) siRNA administered at the same concentrations. The effect of treatment with siRNAs on sensitivity to cisplatin (at 0-64 μM) also were evaluated. FIG. 15 shows that, as the concentration of the 2 siRNAs was increased, the amount of cisplatin required to cause 100% inhibition of the tumor cell viability decreased—from ˜32 μM in the presence of 0.45 nM NS siRNA to <1 μM in the presence of 0.45 nM of BCLxL/MCL1 siRNAs.

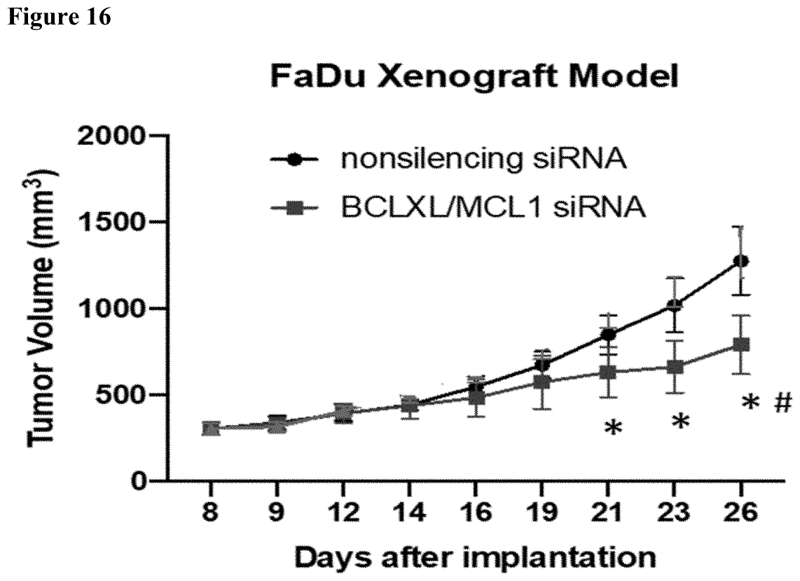

The combination of siRNAs against BCLxL and MCL1 also was shown to inhibit xenografts of H&N cancer when administered intratumorally. An H&N tumor xenograft was generated in mice by injecting FaDu H&N cancer cells into the flanks of the animals (10⁵ cells per animal). After the tumors reached 200 mm³ (day 8 in FIG. 16), BCLxL/MCL1 siRNAs formulated in HKP(+H) nanoparticles were administered BIW (twice per week) in 80 μl per injection at 1 mg/kg into the tumors of the animals. As shown in FIG. 16, the coadministration of the two siRNAs results in a significant reduction in the tumor growth rate in this model compared with a similar formulation that used non-silencing (NS) siRNAs as a control. * p=0.05 between Test and Control values.

In addition, the effect of altering the ratio of MCL1 siRNA to BCLxL siRNA to obtain the best efficacy was evaluated. The data shown in FIGS. 13 and 14 demonstrate that the optimal ratio was obtained at a 3:1 ratio of MCL1 siRNA (seq #4, SEQ ID NO:12) and BCLxL siRNA (Seq #5, SEQ ID NO:5) in FaDu cells—resulting in an IC₅₀ of ˜0.2 nM (FIG. 13). A ratio of 2:1 resulted in a lower inhibition (IC₅₀ of 0.35 nM) very similar to that produced at a 1:1 ratio (0.31 nM).

1. A nanoparticle composition comprising a BCLxL-silencing amount of an siRNA molecule that targets BCLxL and an MCL1-silencing amount of an siRNA molecule that targets MCL1, wherein said siRNA that targets BCLxL is selected from the group consisting of SEQ ID NOs:1-8 and the siRNA that targets MCL1 is selected from the group consisting of SEQ ID NOs:9-13.

2. The composition according to claim 1, wherein said siRNA that targets BCLxL is selected from the group consisting of SEQ ID NOs:1, 4, 5, 7 and 8 and said siRNA that targets MCL1 is selected from the group consisting of SEQ ID NOs:10-13.

3. The composition according to claim 1, wherein said siRNA that targets BCLxL is SEQ ID NO:5.

4. The composition according to claim 3, wherein said siRNA that targets MCL1 is SEQ ID NO:10 or SEQ ID NO:12.

5. The composition according to claim 1, wherein the nanoparticle comprises an HKP.

6. The composition according to claim 1, wherein the HKP is HKP(+H).

7. The composition according to claim 1, wherein the ratio of the siRNA that targets MCL1 to the siRNA that targets BCLxL is about 1:1 or more.

8. The composition according to claim 7, wherein the ratio is from about 1:1 to about 3:1.

9. The composition according to claim 8, wherein the ratio is about 2:1 to about 3:1.

10. The composition according to claim 9, wherein the ratio is about 2:1 or about 3:1.

11. A method of treating a cancer in a subject suffering from said cancer, comprising administering to said subject an effective amount of a composition according to claim 1.

12. The method according to claim 11, wherein said cancer is selected from the group consisting of head and neck cancer, bladder cancer, pancreatic cancer, cholangiocarcinoma, lung cancer (NSCLC and SCLC), colon cancer, glioblastoma, breast cancer, gastric adenocarcinomas, prostate cancer, ovarian carcinoma, cervical cancer, AML, ALL, myeloma, and non-Hodgkins lymphoma.

13. The method according to claim 11, wherein said composition is delivered systemically or intratumorally.

14. The method according to claim 11, further comprising administering an effective amount of a chemotherapy drug.

15. The method according to claim 14, wherein said chemotherapy drug is a platinum-containing drug.

16. The method according to claim 15, wherein said platinum-containing drug is cisplatin, oxaloplatin, or carboplatin.


SEQUENCE LISTING (13 sequences)

SEQ ID NO: 1 - DNA, 25 bp, Artificial Sequence
  Description of combined DNA/RNA molecule synthetic oligonucleotide
ccuacaagcuuucccagaaaggaua

SEQ ID NO: 2 - DNA, 25 bp, Artificial Sequence
  Description of combined DNA-RNA molecule synthetic oligonucleotide
cccagugccaucaauggcaacccau

SEQ ID NO: 3 - DNA, 25 bp, Artificial Sequence
  Combined DNA-RNA molecule synthetic oligonucleotide
ggagccacuggccacagcagcaguu

SEQ ID NO: 4 - DNA, 25 bp, Artificial Sequence
  Description of combined DNA-RNA molecule synthetic oligonucleotide
cggggcacugugcguggaaagcgua

SEQ ID NO: 5 - DNA, 25 bp, Artificial Sequence
  Description of combined DNA-RNA molecule synthetic oligonucleotide
gcguggaaagcguagacaaggagau

SEQ ID NO: 6 - DNA, 25 bp, Artificial Sequence
  Combined DNA-RNA molecule synthetic oligonucleotide
gcguagacaaggagaugcagguauu

SEQ ID NO: 7 - DNA, 25 bp, Artificial Sequence
  combined DNA-RNA molecule synthetic oligonucleotide
ccuugugaagaugauauacuauuuu

SEQ ID NO: 8 - DNA, 25 bp, Artificial Sequence
  combined DNA-RNA molecule synthetic oligonucleotide
ggugaaagugcaguucaguaauaaa

SEQ ID NO: 9 - DNA, 25 bp, Artificial Sequence
  combined DNA-RNA molecule synthetic oligonucleotide
gcugggauggguuuguggaguucuu

SEQ ID NO: 10 - DNA, 25 bp, Artificial Sequence
  Combined DNA-RNA molecule synthetic oligonucleotide
gcuaacaagaauaaauacaugggaa

SEQ ID NO: 11 - DNA, 23 bp, Artificial Sequence
  Combined DNA-RNA molecule synthetic oligonucleotide
gcaaccacgagacggccuudtdt

SEQ ID NO: 12 - DNA, 23 bp, Artificial Sequence
  Combined DNA-RNA molecule Synthetic oligonucleotide
gggauggguuuguggaguudtdt

SEQ ID NO: 13 - DNA, 23 bp, Artificial Sequence
  Combined DNA-RNA molecule synthetic oligonucleotide
uaacaccaguacggacgggdtdt